(19) United States
(12) Patent Application Publication
(10) Pub. No.: US 2022/0152260 A1
(43) Pub. Date: May 19, 2022

(54) AIR PURIFIER

(71) Applicant: Rui FERNANDES, Albufeira (PT)

(72) Inventor: Rui FERNANDES, Albufeira (PT)

(21) Appl. No.: 17/310,616

(22) PCT Filed: Nov. 11, 2019
(86) PCT No.: PCT/IB2019/059658

§ 371 Date: Aug. 13, 2021

(30) Foreign Application Priority Data
Mar. 5, 2019 (PT) 115355

(51) Int. Cl. A61L 9/00; B01D 46/00; B01D 53/26

(52) U.S. Cl. CPC A61L 9/00; B01D 46/0028; B01D 2279/40; A61L 2209/14; B01D 53/26

(57) ABSTRACT

The present invention relates to an air decontamination process system, which comprises one or more depollution towers (T), wherein each tower (T) comprises one or more air inlets, with or without air filtration (2), containing such an air decontamination zone, suitable devices for this purpose, an external tower (3) or air tank, where the air is already decontaminated and treated, and an outlet of decontaminated air at the top of the tower (4).

The air thus decontaminated may be released into the atmosphere, the process of the invention being able to give quantities of unpolluted air of the order of 400,000 m3 to 600,000 m³ per tower (T) per hour.

In this way, the present invention is part of the technical field of the new technologies for the treatment of environmental components, in this specific case, for the treatment and improvement of large volumes ambient air quality.

### TECHNICAL DOMAIN OF INVENTION

The present invention relates to a system and process for depollution of large volumes of atmospheric air, being this system formed by one or more depollution towers, wherein each tower comprises at least one air inlet, and an external structure or air tank, where the unpolluted air is located, which was treated by contacting with crushed pure rock salt, located in a basement of air decontamination, and an outlet for air already treated at the top of the tower so that the unpolluted air can be released into the atmosphere.

So, the present invention is in the technical domain of new technologies for treating environmental components, in this specific case, for treating large volumes of air and improving ambient air quality.

### BACKGROUND OF THE INVENTION

Atmospheric pollution refers to changes in the Earth's atmosphere likely to cause an environmental or human health impact through contamination by gases, solid particles, and liquids in suspension, biological material or energy.

Air pollution can be defined as the presence of substances from human activities or nature itself, which are putting at risk the quality of life of living beings. Polluted air is causing serious problems for man and other beings, as it is becoming improper and harmful to life in general.

The influence of polluting substances on the degree of pollution depends on their chemical composition, concentration on air mass or even depending on climatic conditions, which may influence their dissipation, or mechanisms related to reactions that may give rise to new pollutants.

Air pollutants can be divided into main two groups: primary pollutants and secondary pollutants. Primary pollutants are those that are emitted directly by a source of pollution, such as a car. Secondary Pollutants are those that undergo chemical reactions in the atmosphere, that is, that are formed from the interaction of the environment with the primary pollutant.

Among the main air pollutants, we can mention: smoke, inhalable particles, sulphur dioxide, ozone, nitrogen dioxide, carbon monoxide, methane and others. These substances in excess in the atmospheric air can cause serious damage to the health of the man and living beings on the Planet.

Carbon monoxide, for example, lowers the blood's ability to carry oxygen through the body and can cause residual hypoxia. Ozone, on the other hand, has an oxidizing and cytotoxic role, which can cause eye irritation and decrease lung capacity, for example. Sulphur dioxide is associated with irritations in the upper airways, as well as nitrogen dioxide, which can also cause serious damage to the lungs. Particulate matter (PM) present in air pollution comprises fine particles of suspended solids or liquids. This material occurs naturally from volcanic eruptions, sandstorms, fog formation, and other natural processes. The action of man produces MP from industrial activities, extraction of ore and combustion of fossil fuels, among others. In the atmosphere, these materials cause health damage. The smaller the particle size, the greater the harmful effects caused.

Also, according to the US Environmental Protection Institute (EPA), particulate matter (PM) can be divided into two categories:

the PM-2,5 is made up of particles whose size reaches up to 2.5 micrometers and can be found in fog and smoke.

The PM-10, with particles of 2.5 to 10 micrometers in size, can be found in regions close to industrial zones.

Among others, some of the harmful effects of PM are respiratory and heart problems.

According to a World Health Organization (WHO) study in 2014, air pollution has killed more than 7 million people worldwide by 2012, killing more than AIDS and malaria combined.

The Quality of Donation (IQA) is a standardized indicator of the level of air pollution in a given area, and results from a calculated arithmetic mean for each indicator, according to the results of several zone measurement network stations. The IQA measures mainly the concentration of ozone and particles at the ground level and may include measurements of SO2 and NO2. The parameters of the indices vary according to the regulatory entity that defines them, and there may be several differences.

The conversion of analytical and scientific data into an easily understood index allows the general population to have easier and more comprehensible access to information. The evolution of the IQA is usually made available in real time, especially in the case of large urban or industrial clusters. It is currently a widely used tool in the world as a method of controlling air quality, as well as a means of disseminating scientific information to the community, in an easily understandable way. The data collected by the meteorological stations are treated and inserted in computer programs of atmospheric dispersion modelling, where chemical and physical models of dispersion are applied to predict the chemical and physical behaviour of pollutants and atmospheric mass using scientifically validated models such as CHIMERE. Subsequently, the data resulting from the modelling are cross-checked by those collected by the measuring stations, and then validated or not, according to the degree of agreement. These validated data are taken into account for the overall assessment of the overall state of the air quality level, which is important for verifying compliance with or non-compliance with applicable standards and legislation.

There are several air pollution control technologies and strategies available to reduce air pollution, the following technologies being most used in industry and the automotive sector to reduce the emission of pollutants:

Cyclones of dust are mechanical separators of particles, where the gas with particles is forced to spin in a cyclonic way, causing that through the difference of mass between the particles and the gas they move towards the outside of the vortex, be collected;

Electrostatic precipitator: industrial equipment used in the collection of particulate exhaust gases. These function by electrostatic ally charging the particles and then capturing them by electromagnetic attraction. They are machines of high cost and energy consumption, however, of high efficiency;

Activated Carbon: Activated carbon filters are commonly used in gas purification to remove vapours from oils, odours and other hydrocarbons from the air. Activated carbon is a form of carbon that has been transformed to make it extremely porous and therefore to have a large area available for absorption or chemical reactions;

Catalytic Converter: A device used to reduce the toxicity of exhaust emissions from an internal combustion engine. Introduced in the USA as of 1975 to comply with the EPA's required legislation on harmful gas emissions;

Bio filters: they consist of the application of microorganisms, including bacteria and fungi that are immobilized in a bio film to degrade the polluting compounds. These microorganisms will oxidize matter (CO2 and H2O), thus eliminating unwanted compounds. It is particularly used in the control of VOCs, H2S, odours and ammonia.

Another approach aimed at controlling the harmful effects of pollution, but not to control the emission of pollutants into the atmosphere is Halo Therapy.

Halo Therapy is a type of Alternative Therapy treatment that consists of the therapeutic use of mines and rock salt caves or other forms of exposure to a saline atmospheric environment. The natural mineral deposits of halite are consequences of the evaporation of old lakes and seas. The non-refined mineral salt of sodium chloride also includes varying concentrations of mineral salts such as calcium and magnesium, manganese and sulphates, which have additional therapeutic properties, depending on the source. The special characteristics of the microclimate of a rock salt mine include stable air temperature, humidity and no pollutants in the air, and are unique to each mine.

There are records of improvements in respiration in miners in Roman and medieval times. The Doctor. Feliks Boczkowski—a physician at the Wieliczka salt mine in Poland—wrote in 1843 that the miners of this mine did not suffer from lung diseases and his successor installed a SPA based on these observations. The modern application of this therapy began in Germany when Dr. Karl Hermann Spannagel noticed improvements in the health of his patients after they hid in the Kluterthohle Karst mine to escape bombing during the war. At present it is practiced in places like Low Tatras in Bystrianska in Slovakia, in Wieliczka in Poland, in Solotvyno in Ukraine and many other places.

In Portugal, Louie's salt mines were visited by Portuguese and foreigners for therapeutic reasons. A project for 5/10 years had been a project to build a small hotel/sanatorium for patients with asthma and other respiratory diseases, to treat these illnesses while sleeping, and can resume normal activities during the day. It has been scientifically proven that in a gem-salt mine, such as in a gem-salt gallery, for simultaneous treatment of many patients, the pathogens exhaled by the patients are neutralized by the aerosols of the dried salt rock.

Over time, it has been found that the miners of the rock salt mines, who spend long periods of their life exposed to the environment of these mines did not suffer from non-contagious respiratory diseases.

Gem-salt therapy has been used for centuries in the treatment of respiratory diseases beginning with Hippocrates in Ancient Greece and continued in large scale salt-mines in the twentieth century, particularly in the former Eastern Europe and USSR countries, perhaps because the only way to cure inflammations of the respiratory system of people who, as a consequence, no longer have the symptoms of chronic diseases without side effects, harmful to the body that comes with taking medication.

A study conducted at the Weiliczka rock salt mine in Poland, available at www.Wieliczka-saltmine.com, at the Loulé mine in Portugal at www.infobarlavento.pt and in England at www.saltcave.co.uk and www.saltchamber.com, it was possible to verify that relief of respiratory pathological symptoms was immediate or very rapid and resulted from people's exposure to the environment of these rock salt mines, with dosages of about 50 mg/m³ of air generally being used, in rooms for treatment of respiratory diseases.

The specific construction of the Wieliczka Salt Mine Towers provides a concentration of at least 16% NaCl in the water. The process begins with thickening and increasing the concentration of the brine as it flows through the bramble bushes, and partially evaporates by saturating the air around the building with a saline aerosol. The Tower consists of a cement basin that is located under the wooden construction of the Tower walls, the brine being collected in this basin and then gravitationally channelled to an enclosed underground reservoir situated in a zone near the pumping station. The brine is then conveyed to a system of wooden gutters placed along the top of the walls and at the base of the Tower where it continues to flow through locking valves of wood, regulating the flow of the brine to smaller gutters with cuts, through which it is possible to evenly distribute the brine in the branches of hawthorn under the gutters thus forming the saline aerosol. This aerosol has unique treatment properties, as it has the capacity to penetrate the mucous membranes of the respiratory system and the skin, and the respective process is a natural and specific process of this salt mine, because it works with local material such as wood, the branches of hawthorn and the salt of this mine. However, in order to use this aerosol, users are obliged to travel to Poland.

The same principle is applied in the salt chambers designed and distributed by the company Krysztalowy Swiat since 1999, which comprise a frame, boughs of hawthorn and salt crystals from the Wieliczka salt mines. These chambers are small enough to be used for domestic use, depending on the individual acquisition of the users concerned.

Halo generators are used to reproduce the saline atmosphere of the rock salt mines anywhere in the world. These machines grind salt into microscopic particles, ionize the particles and release them into the atmosphere. Particles with sizes between 0.1-2.5 microns are able to escape the natural defences of the upper airways and reach the lungs at the level of the alveoli. They are typically used in a small room with floor and walls lined with salt.

Methods and apparatus for halotherapy are known in the art as for example those described in US201615266482, CN201610849868, US19970841132, WO2001US05591 and CZ19990003755. All such apparatus includes a vessel for the salts and an aerosol former from that salt. However, personal use devices are impractical to implement in large dimensions and volumes of air.

DE20081016232 discloses an apparatus and method for producing aerosols from salt crystals for simulating the Dead Sea environment, applicable in halo therapy, which comprises a highly concentrated, highly concentrated sea water delivery device like of the Dead Sea described in this document. It includes heating blocks, salt reservoirs and ventilators, in order to obtain the natural conditions of the Dead Sea, in particular a daytime temperature of 35° to 45° C., relative humidity in the area of recovery and healing, i.e. in the climatic zone of 18% to 43%, air saturation with Dead Sea salt ions, such as K1, Ca+2, Br+, Na+, Cl+, Mg+2, its concentration being from 11 to 1000 ion pairs/1 cm³, favourable infrared radiation spectrum with a wavelength of 10-750 nm; light spectrum of visible light predominantly green-yellow in colour with a wavelength of 550-750 nm and air saturation with aroma of essential oils.

The upward air currents formed by the fans create a special natural phenomenon, particularly in the damping funnels, in the form of a constant increase in the layers of air. When the aromas of the essential oils and mineral vapours of the water enter these flows, volatilizing, thus creating the microclimate favourable to the halo therapy. Although this apparatus is already larger in size than previously described, it is also impracticable to implement it in sufficiently large dimensions to be able to clean large volumes of urban and/or industrial air as described in the present invention.

In short, none of the methods and apparatus described above is suitable for promoting large-scale atmospheric decontamination through its installation in urban and/or industrial areas in order to provide high quality air to the populations and in accordance with legal standards for each of these areas.

The present invention proposes a way of arriving the air already treated with rock salt and therefore decontaminated, to the populations and environment, in particular in urban and/or industrial areas of known polluted environment, from the construction of large air towers where the polluted air comes into contact with pure crushed rock salt, releasing it in large quantities.

### DESCRIPTION OF THE FIGURES

FIG. 1 represents a preferred embodiment of the air decontamination system of the present invention, more specifically shows some details of a Tower (T) in which:

1. Represents a polluted air intake in the depollution tower) T);

2. Represents a basement or zone treatment of polluted air;

3. Represents the external structure (in this case the visible part of the tower (T), where the air is already treated and decontaminated;

4. Represents a depolluted air outlet;

5. Represents an already decontaminated air outlet valve or port

6. Represents a set of photovoltaic panels for energy supply to the tower.

FIG. 2 represents another preferred embodiment of the air decontamination system of the present invention, more specifically shows the detail of a tower (T) in which further:

F. Represents the filtration zone of the air entering through the single inlet of polluted air (1);

S. It represents a set of serpentines (7) where polluted but already filtered air is successively injected with the crushed rock salt during its course in the serpentines;

G. Represents part of the devices for crushing pure rock salt:

D. Represents the ground floor of the air decontamination complex, with covers made of transparent material, for example in glass or acrylic material in order to let the sunlight pass to the ground floor, which is at ground level to be able to be visit;

E. Represents a decorative element of the anti-pollution tower. The crushed rock salt to be injected during the course of polluted air in the serpentines located in the rear zone and the remaining lateral parts of the zone of implantation of the air decontamination system, which are not visible being marked in this figure, only in the posterior zone however, four injection zones of the crushed rock salt arranged along the perimeter of deployment of the tower, accompanying the circulation of the polluted air in the serpentines (S) of the tower (T).

FIG. 3 shows another preferred embodiment of the air system of the present invention, more specifically showing the deployment of two towers (T1 and T2) in an area with urban and recreational purposes.

### DESCRIPTION OF THE INVENTION

The present invention relates to the development of a system and a process for the decontamination of atmospheric air.

In the context of the present invention, the term “rock salt” defines a sodium chloride-based material, accompanied by potassium chloride and magnesium chloride, which occurs in deposits on the earth's surface. This material of natural origin belongs to the group of sedimentary rocks, more specifically to the group of sedimentary rocks chemogenic, evaporites, to be formed by chemical reactions. Thus, this term is applied to the salt obtained by chemical precipitation by evaporation of water, which explains the name evaporites, from ancient marine basins in sedimentary environments. The rock salt does not react with acids, has non-metallic luster, scratches or white lines and has no cleavage.

The atmospheric air decontamination system comprises one or more air-cleaning towers (T), the number to be implemented in the system of the amount of air to be treated.

Each tower (T) comprises an air inlet (1), a basement (2), or a polluted air treatment zone, by the contact of this air with crushed rock salt, an external structure (3) or depolluted air tank, thus already treated and at least one depolluted air outlet (4).

In a preferred embodiment of the invention there is a return valve (5) or outlet port at the top of the outer structure (3) which has the function of opening to let the treated air pass through to the atmosphere, and if necessary close working in a continuous process.

Discharge of air treated to the atmosphere is done at a rate which is similar to the rate of air treatment, i.e., air continuously entering the system through the single air inlet (1), being treated in the basement (2) by contact with the crushed rock salt, passing to the outer structure (3), where it is expelled from the tower (T) immediately by the outlet (4), remaining in the tower only during the period of time when it is injected the salt—(G) along the serpentines at 200, 300, 400 or more meters of their course in the serpentines (S) and (7).

In another embodiment of the invention the inlet (1) and outlet (4) flow rates, i.e., and the air quantity to be treated is controlled by the volume of the single active/operating air inlet (1). If it is desired to increase the amount of depolluted air to be expelled into the atmosphere, the volume of the polluted air inlet (1) is increased. If it is desired that a certain tower (T) of the Invention system functions with a larger capacity, it is enough to enter a larger volume of air to be treated.

It is not expected that there will be large fluctuations in the volume of air to be treated, the air-circulating pumps of which operate at a speed recommended by their manufacturers, as well as the technology to be used, either mobile or the control of crushing of m³, which adapts to the amount of airborne pollution to be treated so that the system will last for five to ten years with routine maintenance only.

The outer structure (3) is preferably built in cement and iron or equivalent materials, implanted at ground level, above a structure having a function of tank (s) or base (2). The elements of the rock salt crushing devices capable of crushing into particles of suitable dimensions between 2 and 5 micrometers are installed in the serpentines (S) and (7) on the sides of the tower T, around.

Between the external structure (3) of the tower (T) itself and the base (2), underground there may be a multipurpose ground floor for example for recreational, cultural use, and a small part of that area may be used to store salt—pure gem, for technical flooring, maintenance workshop and other storage.

Each tower (T) can contain several rock salt crushing devices, functioning interconnected in sets of 6 and 12 crushers. For easy maintenance by replacing the whole assembly, which may have in each tower 10, 20 or more sets of 6 or 12 rock salt crushers located in the lateral zones along the polluted air path in the coils (S) and (7) of the tower air decontamination system in the basement (G), along with the circulation of polluted air in the coils. The total number of such devices depends on the planned quantity of rock salt to be crushed, which will be dimensioned in durability and effectiveness to run continuously, night and day for years.

The rock salt grinding devices of the present invention are preferably produced in stainless steel or equivalent material, resistant to the rock salt abrasive, so as to be able to crush and grind the pure rock salt into micro particles of variable size from 0.1 to 10 micrometers in diameter, preferably 1 to 7 micrometers in diameter, even more preferably 2 to 5 micrometres in diameter, these values corresponding to the indicative average reference indices respirable by humans and animals.

The contact between the crushed salt and the polluted air is done by injecting it into the polluted air circulating in the serpentines (S) and (7), which are placed on all sides of the tower in the basement in order to enhance the treatment of the volume of air that will be in contact with the rock salt in the basement (2), thus resulting in the elimination of the pollution of the treated atmospheric air. The treated air is then conveyed to the outer structure (3) and released/expelled into the atmosphere via the air outlet (4).

In a preferred embodiment of the invention, the proportion of rock salt to be contacted with polluted air for treatment and depollution purposes may range from 25 to 200 mg of rock salt per m³ of polluted air to be treated, preferably 50 to 100 mg of rock salt per m³ of polluted air to be treated.

The system operates in a closed and airtight circuit. The polluted air enters the filtration zone of the airborne dust, including metal for the serpentines (S) to be injected the crushed salt into micro particles being unpolluted in its circulation for 100 to 400 meters of the serpentines (S). The pumps that push the air into the tower and filter the suspended dust, with liquids or other filters that give it the necessary pressure for the air to circulate in a closed circuit, passing through the injectors of rock salt that throw it in the air polluted until its exit.

The decontamination process must thus be “SLOW” for the air to circulate in the serpentines continuously and reach the end in 5 to 15 minutes and be discharged into the atmosphere in volumes of about 400,000 to 600,000 m³/hour/tower.

In another preferred embodiment of the invention there is also provision for liquid particulate filters capable of retaining the particulate material (MP), i.e. fine particulate pollutants of solids or suspended liquids which are present in the polluted air entering the system (1). These liquid-based filters, other filters and magnets may be located at a lateral air inlet in the tower (1), so as to free them from these particles to then enter the serpentines (S) and (7), always in hermetically controlled circuit where the rock salt is injected. The entire process of air decontamination with an average duration of 5 to 15 minutes in continuous mode can run day and night for several years.

Suitable filters within the scope of the present invention may be liquid-based filters, HEPA (Efficiency Particulate Arrestance), membrane filters or the like or a combination of more than one type of filters in order to be able to efficiently separate the particles in the air to be treated, according to their size and quantity depending on the type of pollution to be removed.

In a preferred embodiment, the system of the present invention uses liquid and HEPA filters composed of a randomly placed fibber mesh. The fibbers comprise glass fibber having diameters between 0.5 and 2æm.

In another preferred embodiment the system of the present invention may use membrane filters, preferably of polymer membranes, more preferably membranes comprising mixtures of polymers such as poly (N-isopropyl acryl amide) [PNIPA] (and polyethylene glycol) [PEG], comprising for example 0, 2, 4 and 8% mm PEG.

Still further preferred, the system of the present invention comprises the use of at least one liquid filter and a HEPA or membrane filter.

Optionally, each tower (T) further comprises PV panels (6) for example placed around the outer structure (3) for supplying power to the system of the invention.

In another preferred embodiment of the invention there is also provided the existence of energy accumulators which allow the solar/photovoltaic panels to receive energy during the hours of solar luminosity and to retain it, possibly releasing it during the period of darkness or when required.

The energy supplied to the system may, in addition, be supplemented by supplying energy from other types of power supply, such as from the electrical network or from an electric generator. In this way, in case of prolonged failure of the photovoltaic components or other type of necessity or emergency, it is thus possible to keep the system in operation until repaired.

Thus the energy for the rock crushing devices, pumps, electric motors, air and liquid filters and all other equipment including computer control of the programming technology of the rock-salt crushing sets and other equipment is preferably supplied by photovoltaic panels (6) or equivalent elements, placed around the towers with accumulators of energy to continue to have energy at night, i.e. even without the presence of sunlight.

In this way each tower (T) will comprise solar panels (6) located for example around the outer structure (3) and the ground floor roof, if necessary.

The outside of each tower (T) can be approximately 20 to 50 meters high, 30 to 60 in diameter at the base at ground level, tapering at the top for 5 to 15 meters in diameter with a valve (4) and 5) at the top of the outer structure (3), to exit the unpolluted air and to close the outlet if necessary.

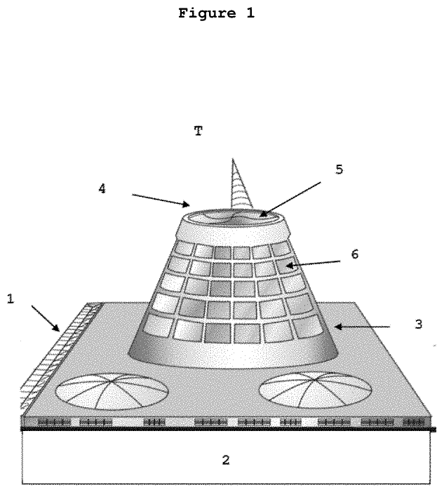

Preferably the outer structure (3) of the towers (T) has a round pyramidal shape of about 20 to 50 meters in height, 30 to 60 in diameter i.e. tapering at its top to 5 to 15 meters and may contain therein between 50 to 56,000 m³ of unpolluted air (FIGS. 1,2 and 3).

Preferably the base (2) of the towers (T) has dimensions of about 80×80×20 meters with a volume of about 128,000 m³, in addition to the external structure (3), the technical ground floor and other constructions having to reach an area of construction of about 25,000 to 30,000 m³, of slabs with 20 to 30 cm of thickness being able to adapt the measures in each case.

Advantageously, the towers (T) of the present invention are installed in the vicinity of urban and/or industrial areas of polluted air to be treated, even more preferably in an integrated manner in the environment, taking their location into account the prevailing winds for a faster dispersion of unpolluted air over polluted areas from 6 to 11 km.

The air decontamination system is controlled through a computer program, wherein the scheduler of each set of grinding devices includes a memory board of its control, which allows a lower concentration of dry aerosols, based on a suitable microprocessor, the respective memory board comprising a database of high accuracy measurements of the concentration of dry aerosols in the serpentines (S) and (7) of the large air tanks 2 controlled by real-time displays preferably installed on the ground floor which can be opened to visitors.

The air treated according to the process of the invention exhibits characteristics similar to that naturally occurring within the rock salt mines and can be continuously released into the atmosphere for several years, with beneficial effects on the environment, humans, fauna and flora.

The air decontamination process of the present invention comprises contacting the polluted air to be treated with more specifically crushed rock salt, the process of the invention is carried out in at least one depollution tower (T) as previously described wherein:

a) Air to be treated between air inlets (1), preferably in a single air inlet of a depollution tower (T);

b)—the air to be treated is filtered and taken to a basement where its contact with the crushed rock salt is promoted;

c) The treated air resulting from step b) is conducted into the atmosphere passing through the outer structure (3) of the tower (T) through a single air outlet (4).

Preferably the polluted air entering the tower (T) in step (a) is filtered prior to meeting the crushed rock salt, through at least one air filter capable of holding the polluted air material.

Preferably the size of the crushed salt particles to be contacted with the filtered air in step (c) ranges from 0.1 to 10 microns in diameter, preferably 1 to 7 microns, even more preferably 2 to 5 microns in diameter.

Preferably, the crushed salt meets the polluted air entering the previously filtered system or without prior filtration in the serpentines (S) and (7).

In a preferred embodiment of the invention the proportion of rock salt to be contacted with the polluted air for the purposes of treatment and depollution of such air varies from 25 to 125 mg of rock salt per m³ of polluted air to be treated, or even more preferably from 50 to 125 mg of rock salt per m³ of polluted air to be treated.

The air remains in contact with the crushed rock salt for a period of time ranging from 5 to 10 minutes, preferably from 5 to 15 minutes or further from 5 to 20 minutes, depending on that period of time of the volume of air, the bombs pump to the air inlet of the tower, so that the air circulation velocity in the serpentines is what is necessary to obtain the desired results: the air enters polluted and leaves at the top of the tower unpolluted, adjusting itself pumping at the air circulation velocity, to achieve such an objective.

The ideal relative humidity of the air to be treated should be approximately between 35% and 65%, on average 50% possible to control with the assembly of an electric air heater/dehumidifier, for example on the top of the air filter (F).

The air circulation in the towers is automatically performed from the air pumped to the entrance of the polluted air in the system that feeds it continuously throughout the entire process of air decontamination.

The air decontaminated may be released into the atmosphere and the process of the invention may origin quantities of unpolluted air of the order of 400,000 m³ to 600,000 m³ per tower (T) per hour.

In this way, it is possible to obtain quantities of depolluted air that can reach 600.000 m³ of air treated by each tower per hour, about 14.000.000 m³/day X (number of towers)×10 years containing on average 50 mg of rock salt, reduced from 0.1 to 10 microns in diameter, preferably from 1 to 7 microns, even more preferably from 2 to 5 microns in diameter, suitable for human and animal breathing, and also for the flora.

The rock salt crushed in respirable micro particles is then filtered and injected into polluted atmospheric air capable of eliminating its polluting elements in accordance with the European Standards for bactericidal efficacy EN1276 and EN1040, fungicide EN1275, viricide AFNOR 72180, sporicidal NFT72.230, and may be used without limitation in any medical practice in any EU country without any harmful or secondary effects, for humans and animals and for life in general (fauna and flora), including pathogens exhaled by patients (in the case of the salt mines and galleries) neutralized by dry-salt aerosols.

The treated air has been shown to be beneficial to treat or ameliorate respiratory conditions notably asthma, shortness of breath, chest tightness, chronic bronchitis, allergies and respiratory infections of industrial and domestic pollutants, conditions due to smoking, colds and flu, adenoids, wheezing sore throat, rhinitis, pharyngitis, sinusitis, thoracic oppression, tonsillitis, hay fever, sleep apnoea, ear problems, smoker cough, dry cough and cough with or without expectoration, mucosal oedema, pneumonia after acute phase, rhino sinusopathy, smoking, as well as the dermatological forum such as acne, eczema and psoriasis, or general conditions of stress, fatigue and depression. It also reduces the pollution inside the houses depending on the effectiveness of the same, reinforcing the immunological apparatus being less violent diseases.

### EXAMPLES

### Example 1—Construction of an Air Decontamination System with a Tower (T)

Excavation of the deployment site of the system to house the base tank in the basement (2) with a volume about 180,000 to 200,000 m³. Construction of the outer structure (3) of the towers (T) having a round pyramidal shape, about 20 to 50 meters high, 30 to 60 meters in diameter at its base at ground level, i.e. tapering at its top to 5 to 15 meters in order to contain between 50 and 56,000 m³ of decontaminated air. (FIGS. 1, 2 and 3).

In this embodiment of the invention the air entered in (1) is filtered in (F) through liquids, HEPA filter or others, circulating in the basement (2) in 5 compartments, referred to herein by serpentines with 5 meters in height and 75 meters each of about 400 meters to be treated in this course, by injecting pure rock salt reduced to dimensions of 2 to 5 micrometers until it enters the outer structure (3) of the tower (T) with about 50,000 to 56,000 m³ of volume, and exit at the top of the tower (4), (T), into the atmosphere.

### Example 2—Construction of an Air Decontamination System with Two or More Towers (T)

In this example two tanks are developed for the treatment of polluted air. Thus, this system has a basement (2.1) with a volume of the order of 50,000 m³ and a second basement (2.2) with a volume and dimensions as described in example 1, i.e. a volume of 180,000 to 200,000 m³.

### Example 3—Atmospheric Air Decontamination Process

In this embodiment of the invention the decontamination process is carried out in a system as described in Example 1, wherein:

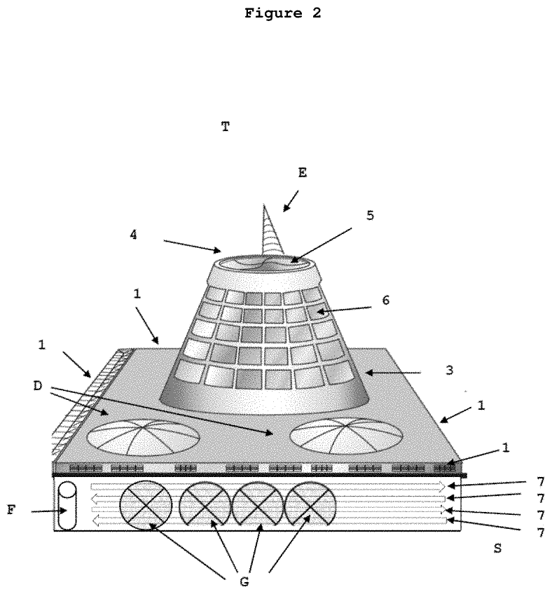

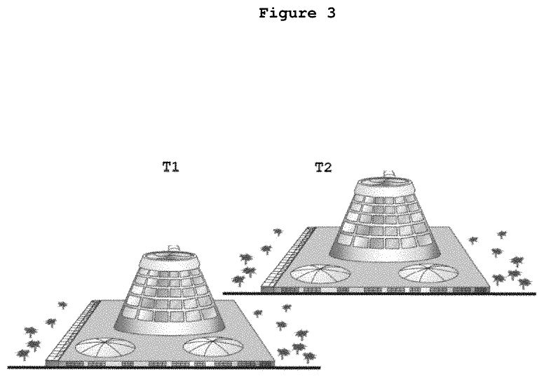

a) The air to be treated enters the tower (T) through the single air inlet (1) which is located at the base of one side of the depollution tower (T), as shown in FIG. 2.

b)—that air is conducted to the basement (2) by first passing through a filtering zone (F) for removal of particulate material, through liquid filters, HEPA or others.

c) then the already filtered air is conveyed to the air treatment system, which comprises the sets of grinding devices of the rock salt (G) and a plurality of serpentines (S) and (7), for contacting filtered air with the crushed salt, in particles of 2 to 5 microns in diameter for about 5 to 20 minutes.

(d) a proportion of rock salt coming into contact with the polluted air is injected for the treatment and decontamination of that air with 50 to 100 mg of rock salt per m³ of polluted air to be treated.

e) the air treated in c) and d) is conveyed to the outer structure (3) and to the air outlet (4) of the tower (T) through which it passes when it is expelled into the atmosphere.

### Example 4—Process for the Cleaning of Atmospheric Air with Metal Particles

In this embodiment of the invention the method of air decontamination takes place in a system as described in example 2. In addition, a strong magnet is placed against the bottom of the water tank to fix metal pollutants.

The water is in agitation.

The air enters the bottom of the water tank, collecting this water by difference in level, using a water injector in the tank to push the dirty water with particles suspended in the air to the outlet of the tank, and through a suction pump will be recycled in treatment plants for watering and other uses.

1. A system for the decontamination of atmospheric air characterized by said system comprising one or more depollution towers (T), each of the towers (T) comprising an air inlet (1) through which the polluted air to be treated enters; a base reservoir (2) where the treatment of polluted air is carried out by contacting this air with crushed rock salt by at least one set of grinding devices of the rock salt (G); and at least one unpolluted air outlet (4), thereby defining a polluted air treatment circuit.

2. A system according to claim 1, characterized in each tower (T) comprises several sets of 6 to 12 crushers, each of which may comprise 10, 20 or more sets of 6 to 12 rock salt crushers located in the four side zones along the polluted air path in the serpentines (S), (7) and (G) for crushing the rock salt capable of running continuously.

3. A system according to claim 1, characterized in each rock salt crushing device is made of a resistant material because the rock salt is abrasive and is capable of crushing the rock salt into particles of variable size, 1 to 10 microns in diameter, preferably 1 to 7 microns, even more preferred 2 to 5 microns in diameter.

4. A system according to claim 1, characterized in each tower (T) comprises a set of serpentines (S) and (7) where the contact of the crushed rock salt with the polluted air to be treated is promoted.

5. A system according to claim 1, characterized in each tower (T) comprises one or more particle filters (F) capable of holding the particulate material (MP) present in the polluted air through the inlet (1) these air filters are located prior to the start of the air decontamination circuit through the set of crushing devices of the rock salt (G).

6. A system according to claim 1, characterized in each tower (T) comprises an air check valve, if necessary (4) and (5) at the outlet of the outer structure (3) at a pressure of 1 atm.

7. A system according to claim 1, characterized in each tower (T) comprises one or more photovoltaic panels (6) for supplying power to the system.

8. A system according to claim 1, characterized in it comprises energy accumulators to ensure the supply of energy in hours of sunlight.

9. A system according to claim 1 characterized in each tower (T) has a round pyramidal outer structure (3) and a base (2) made of cement and iron.

10. A system according to claim 1 characterized in each tower (T) has an outer structure (3) having a round pyramidal shape of about 20 to 50 meters in height, 30 to 60 meters in diameter at its base at level of the ground tapering at its top to 5 to 15 meters and may contain within its interior between 50,000 to 56,000 m³ of depolluted air.

11. A system according to claim 10, characterized in each tower (T) preferably comprises an external structure (3) having a diameter of about 30 to 60 meters at its base level, tapering at its top to 5 to 15 meters, measured which will allow to contain within its interior a volume of about 50,000 to 56,000 m³ of decontaminated air (4), based on a basement tank for the treatment of polluted air having dimensions of about 80 m×80 m×20 m, a volume of about 128,000 m³, totalling the area of construction of all tower structures about 25,000 to 30,000 m² with slabs 20 to 30 cm thick.

12. A process for the decontamination of atmospheric air characterized by promoting the contact between the atmospheric air and the rock salt, and comprises the following steps:
a) The air to be treated enters a single air inlet (1) of a dehumidifying tower (T) as recited in any one of claims 1 to 11,
b) The air from step a) is treated through the contact with crushed rock salt by at least one of the assemblies formed by grinding devices (G),
c) The treated air resulting from step b) is conducted into the atmosphere, passing through the outer structure of the tower (3) through at least one air outlet (4), as described in any of claims 1 to 11.

13. A process according to claim 12 characterized in the polluted air of step a) is filtered in step b) through at least one air filter (F) before being contacted with the rock salt.

14. A process according to claim 12 characterized by the rock salt is crushed at least one set of grinding devices (G), the size of the crushed rock salt particles of 0.11 micrometers, preferably 1 to 7 microns, even more preferably 2 to 5 microns in diameter.

15. A process according to claim 12 characterized in the air to be treated meets the crushed rock salt in at least one set of serpentines (S) and (7).

16. A process according to claim 12 characterized in the proportion of the air to be treated with crushed rock salt varies from 400,000 to 600,000 m³ per hour, even more preferably 500,000 to 600,000 m³ per hour.

17. A process according to claim 12, characterized in the relative humidity of the air in step b) can be between 35% and 65% in an average of 50%, so that the gem-salt is less abrasive, and may be controlled by use of a dehumidifying device or air heater to be placed at the top of the air filtration zone with or in liquid before it comes into contact with the crushed rock salt.

18. A process according to claim 12 characterized in that it takes place over a period ranging from 5 to 15 minutes, preferably ranging from 5 to 15 minutes, even more preferably from 5 to 20 minutes, depending on the period, of the volume of air that the pumps pump into the air inlet of the tower, so that the velocity of air circulation in the serpentines, adjusting the speed of the pumping to the velocity of air circulation.
